(19) United States
(12) Patent Application Publication
(10) Pub. No.: US 2012/0209136 A1
(43) Pub. Date: Aug. 16, 2012

(54) Method and Apparatus for Determining and Improving Health of an Individual

(76) Inventor: Z. Ming Ma, Shanghai (CN)

(21) Appl. No.: 13/453,969

(22) Filed: Apr. 23, 2012

Related U.S. Application Data
(63) Continuation-in-part of application No. 12/381,293, filed on Mar. 10, 2009.

(51) Int. Cl. A61B 5/053

(52) U.S. Cl. 600/547

(57) ABSTRACT

A method and apparatus for obtaining data relevant to the state of health of an individual by measuring the signal spectra at various Jing Luo network termination points on the individual's body. Illustratively, the measurements are at points on the individual's hand, implemented with a glove that includes numerous electrical point contacts. A healing session ameliorates a malady by identifying the signature of the malady as reflected in a chosen subset of termination point, determining the amount of energy that is necessary to null out the malady's signature, and applying the determined energy to one or more termination points.

This is a continuation-in-part of U.S. patent application Ser. No. 12/381,293, filed Mar. 10, 2009, which is hereby incorporated by reference.

### BACKGROUND

This relates to a method and apparatus for determining and for improving the health of an individual.

Chinese Medicine concepts and practices originated long before the onset of the modern fields of anatomy, physiology, surgery and other invasive diagnostic and healing techniques. Root-cause diagnosis of an illness is based on observations of the exogenous physical symptoms, such as temperature, facial appearance, perspiration, heart-beat, breathing and pulse rate patterns of the patient. Practitioners of Chinese Medicine combine these observations, knowledge that was accumulated through studies and practical experiences, to locate the source and determine the causes of abnormality.

Definition and terminology for the anatomical organs according to Chinese Medicine differ from those in modern physiology even though the domain of the overall coverage is the same. For example, the term “heart” is understood to include the heart, as is in the modern anatomy, and the auxiliary vascular and eletrochemical-network systems. Since the heart cannot function without the support of its auxiliary systems Chinese Medicine implicitly recognizes the potential correlation of pathology of the anatomical heart and that of its auxiliary support systems. Herein, the term “organ” is used in the more encompassing understanding of Chinese Medicine.

It is also a Chinese Medicine concept that communications among the various organs are channeled through a complex network of Jing Luo. A person is expected to be in good physical health when communications flow is unimpeded in the Jing Luo network, whereas a blocked or congested Jing Luo network signifies ailment. Interconnectivity via the Jing Luo network implies that ailment in a given organ can, and usually does, involve other organs.

According to the modern notions of anatomy, the human body comprises the skeletal frame with the attached muscle masses for movement and for mechanical support for other more localized organs, such as the digestive system, the respiratory system, the reproductive system and the urinary system. Interconnecting these localized systems are the cardiovascular system for the internal transport of blood, oxygen, and carbon dioxide, the endocrine system for integration and coordination of hormones, the lymphatic system for immunal regulation, and the nervous system for electrochemical signaling. The term “organ” as used in the more encompassing Chinese Medicine sense comports with the body “subsystem” of modern anatomy notions, and that is the term used herein.

Although Jing Luo has not been identified with a definitive set of physical constituents in the human body, it is nonetheless reasonable to consider it as a virtual network that is capable of channeling signals between, and facilitating communications among organs and other body parts. Plausible constituents for this virtual network include the blood vessels, the nerves, the bones and the muscle masses.

The physics, particularly the electrical characteristics of several of the constituents have been extensively studied and modeled. For example, it is well known to nutritionists that the electrical equivalent for the muscle mass is a complex reactive network of resistance and capacitance, and that the electrical conductivity of blood is akin to a simple conductor. The fact that the muscle reactance can change with the ionic contents of its surrounding environment is also well known to physiologists. Animal studies have revealed that both for large and small animals the electrical impedance of the bone can be characterized by a simple network of resistors and capacitors.

Modeling of the electrical characteristics of the human cardiovascular system against known EKG data in the low frequency range of 120 Hz or below also exists in the literature. In the higher frequency domain up to 1 kHz, EKG (more commonly referred to as High Frequency Electrocardiography) studies pertaining to better detection of Myocardial Ischemia and other coronary artery diseases are a hot research topic. However, I am not aware of any systematic electrical impedance information in the higher frequency ranges, regarding the cardiovascular network, nor the neural networks that interconnect multiple organs.

The physics of signal transmission in a single neuron takes on the characteristics of a complex electrical circuit with interesting features such as switching, tuning, and even resonances. Proper characterization of the neural network related to a given organ, and by inference, that portion of the Jing Luo system, requires analysis of its impedance spectra.

Chinese Medicine generally holds that Jing Luo evidences itself on the surface of the human body. These are referred to as the termination points. In fact, according to several schools of practitioners, a large collection of these termination points are present on the palm. For example, Jing Luo connected to the stomach terminates at the center of the palm whereas the heart evidences itself at the intersection of the backward extension of the thumb and the forefinger of the palm. The lungs are at the base of the fourth finger and the pinky. A correspondingly detailed map is believed to hold with the foot hosting these Jing Luo termination points. The state of an individual's physical health can thus be gleaned via these termination points. See, for example, a detailed description of the twelve main arteries of the Jing Luo network and some of its termination points in Chapter 2 entitled “The Twelve Jing Arteries” in Biological Physics of Acupuncture and Jing Luo, edited by Zhu Zongxiang and Hou Jinkai, Beijing Publisher (1989) ISBN 7-200-00871-0/R.28.

According to the generalized Thevenin's Theorem, any complex network of resistors, capacitors, inductors and signal sources can be reduced to a simple network of impedance and a single signal source when viewed across two points of the circuit. Salient characteristics of the impedance network, such as attenuation, decay and resonances, evidence themselves as voltage spectra at the termination points.

Since communications among all organs are channeled through the same Jing Luo network, impedance, or alternatively, voltage spectra from a multitude of points on the human body will be needed in order to deduce information from any given organ. If one measures the voltage spectrum at a single termination point (relative to a chosen common point) or a cluster of termination points in close proximity to a particular organ, information that one can glean from the data may be mostly from that particular organ with minor interference or contamination from others. Such is the case with EKG or the EEG technologies.

Simply observing voltages at a cluster of termination points is sometimes useful, however, not sufficient to identify maladies in tested individuals, and certainly not to promote healing or amelioration of symptoms.

### SUMMARY OF THE INVENTION

Based on the Chinese Medicine concept of the Jing Luo network, it was realized that the voltage spectral pattern on the hand, or foot, or any of the other areas of the body that contain numerous Jing Luo termination points, can indicate whether the Jing Luo network is impeded in some way or not, and a person's health status can be assessed from the voltage spectral patterns. Accordingly, disclosed is a method and apparatus for obtaining data relevant to the state of health of an individual by measuring the signal spectra at various points on the individual's body. Illustratively, the measurements are at points on the individual's hand, implemented with a glove that includes numerous electrical point contacts, and the signals that are measured are reflective of voltage spectra, relative to a common measuring point.

In addition to determining the state of health of an individual, including identifying specific malady or maladies that the individual has, disclosed is a method and apparatus for application in a healing process that ameliorates the effects of a patient's malady.

### BRIEF DESCRIPTION OF THE DRAWING

FIG. 1 illustrates a glove that may be used in connection with the method and apparatus disclosed herein;

FIG. 2 is a block diagram of an apparatus in accord with the principles disclosed herein;

FIG. 3 is a flow diagram of a method in accord with the principles disclosed herein; and

FIG. 4 is a flow diagram of another method in accord with the principles disclosed herein.

### DETAILED DESCRIPTION

As described above, it is known that the Jing Luo network, as reflected by termination points, can be used to report on the health status of organs. It is also known that electrical activity is associated with many, and perhaps all, organs.

In accord with the principles disclosed herein, the ancient art of Jing Luo is combined with the more recent scientific findings to quantify attributes of the Jing Luo network by measuring voltage spectra at different termination points.

While concrete quantitative assessments are a hopeful goal, it is currently more realistic to set forth a comparative assessment. That is, in accord with one aspect of this disclosure, an individual's Jing Luo is measured, and based on the obtained measurements a determination is made as to whether the individual is probably in good health or, to the contrary, that a part of the body (e.g., the liver) might not be in good health; all based on comparison to a selected norm. In one illustrative embodiment, the Jing Luo is reflected in the time series of voltages that are measured at predetermined termination points on an individual's skin or other accessible surfaces, the measurements are processed to obtain frequency spectra of the measured values, and compared to corresponding information from database, which information is derived from a statistically significant number of individuals of, for example, similar standing (e.g., ethnic background, sex, age), or to data of past measurements of that same individual.

The method of this invention does not definitively state that an individual is well or is not; it is more an indication of probability. In that sense, this is not too unlike a conventional blood test that provides the physician with a plethora of indicators. As in a conventional blood test, where one indicator that is outside the accepted range does not mean that the individual is definitely sick, an impeded Jing Luo does not indicate that the individual is definitely sick.

In order to determine the voltage of any particular point on the health-indicating surface (relative to a selected common point), a contact point (or localized area) needs to be established with each of the particular termination points to which a sensor can be coupled for energy to flow to, or from, the termination point. This may be an actual electrical connection, much like the electric contacts in the case of EKG measurements, or other coupling means. Since in accord with the principles disclosed herein the analysis encompasses frequency spectra, the sensors may include other than physical contact means (e.g., electrostatic, electromagnetic, etc.).

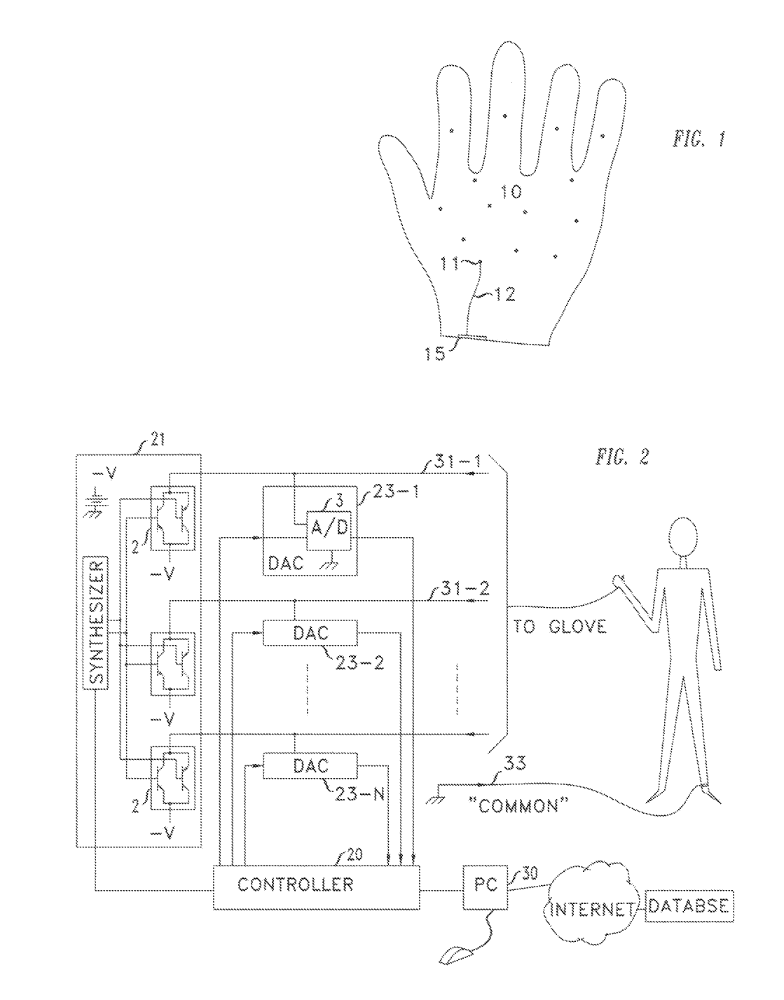

FIG. 1 presents one illustrative embodiment in accord with the principles disclosed herein, where a glove 10 constitutes a Jing Luo Applicator/Detector (JLAD) device. Illustratively, the glove is woven from non-conducting material, such as cotton, and includes a number of electrical contact points (sensors) 11, such as copper rivets affixed to the inside of the glove. The glove is fashioned tight enough so that when wearing the glove, these sensors make good electrical contacts with, for example, the palm and the fingers. A wire 12 is attached between each of the sensors and connector 15 so that the voltage measuring signals can flow to the measuring equipment. For a JLAD device that is used for a foot as a source of Jing Luo network presence, a sock of a similar design is used. It may be noted that glove 10 offers the advantage of not requiring the practitioner to know precisely where on the hand are the preferred termination points, and it also makes it easy to effect a good electronic coupling. In the context of this disclosure, the term “electronic coupling” encompasses all approaches for coupling electrical energy, including, conductive, capacitive, inductive, and electromagnetic. In this, more expanded concept of coupling, the notion of termination point vicinity is more appropriate than the notion of a termination point. Hereinafter, wherever appropriate the term termination point it is meant to encompass the termination point vicinity, or vicinity for short.

FIG. 2 presents an illustrative block diagram of equipment that may be used for a session where healing of a patient's malady is undertaken, or for a session where merely diagnosis of the patient's state of health is assessed.

In a healing session, as disclosed below, voltages are measured at specified termination points of the patient, healing energy that is to be applied to particular vicinities of the patient is computed from the measured voltages, the energy is then applied to the patient, and the process is repeated in a classic feedback manner.

In accord with FIG. 2, whether in the course of a diagnostic session or a healing session, a preselected vicinity of the patient's body is coupled to a “common” point of the FIG. 2 apparatus and one or more sensors are coupled to specific termination point vicinities on the patient's body. Illustratively, the common point is at one of the patient's ankles, and the sensors are coupled by means of the worn FIG. 1 glove. One sensor couples its voltage via line 31-1 to a data acquisition circuit (DAC) 23-1, which contains an analog-to-digital (A/D) converter 3, another sensor couples its voltage via line 31-2 to DAC 23-2, etc. Controller 20 directs collection of voltage samples from specific termination points on the employed JLAD (in this case, the glove) and applies the collected voltages to processing device 30. In the course of a healing session, with the aid of a database that may be remote, device 30 computes measures of energy that are to be applied to one or more sensors of the JLAD (or to a sensor that is not embedded in the JLAD—not shown), and through controller 20, device 30 directs synthesizer 21 to generate and apply the computed energy measures to one or more sensors that are coupled to specific termination point vicinities on the patient's body. In the course of a diagnosis session, information obtained from the database assists in determining whether the patient is in a healthy state of not. In such a session, for example if impedance spectrum is desired, synthesizer 21 may be used to apply a current having a flat spectrum over a given frequency band to the patient's termination points (via the sensors in the JLAD).

Specifically, upon receiving a gating signal from controller 20 at the onset of each sampling cycle, each of the DACs senses and integrates the charges on the respective sensor relative to the “ground” potential over the duration of the gating signal, converts the applied analog signal to digital format, and presents it to controller 20. In applications where it is believed that the signal to the A/D converter contains significant energy at frequencies above half the sampling rate of the DAC, an appropriate filter may be included to precede the A/D converter to prevent contamination. More commonly, the rate at which DAC samples the analog voltage is adjusted to be at least twice the highest frequency of interest. Lastly, the measurements obtained by controller 20 are provided to processor 30 (illustratively, a personal computer) for analysis. Hereinafter it is assumed that a voltage sampled by the A/D converters is appropriately band limited to begin with, or is filtered to be band limited,

Typically it is desirable to detect the signals of all of the sensors at the same instances. The A/D converters are controlled accordingly to capture their respective signals concurrently, and to then compute the digital representations of the captured signals and apply those digital representations to the controller. It is also possible to employ sample-and-hold circuits, instead of individual A/D converters 3, to sample and hold the voltages simultaneously and to apply the analog signals to a single A/D converter that is situated within controller 20 in a seriatim fashion. Obviously, when using a single A/D converter, it needs to be fast enough to accommodate a sampling rate that is appropriate for the highest frequency of interest in one signal, multiplied by the number of sensor signals that are to be converted.

As indicated above, a diagnostic session consists of sensor's being coupled to termination points of the individual, such as by wearing the JLAD device The controller 20 collects the voltage readings of the A/D converters and applies them to PC 30, wherein the signals are processed or, alternatively, are forwarded to another location for processing. Illustratively, the forwarding may be to a remote computer, via the Internet. The processing is preferably carried out for all of the frequencies simultaneously, for example by using the Discrete Fourier Transform (e.g., employing the FFT algorithm) on a time series of voltage samples. The size of the sample set is determined by the desired frequency resolution and the statistical uncertainty of the voltage spectrum, as is well known to experimental scientists.

One purpose of a diagnostic session may be to determine whether the tested individual is likely to be in general good health. Another purpose may be to evaluate one or more particular organs (e.g., heart, liver, spine, etc.). Yet another purpose of a test may be to identify or confirm the presence of a particular malady given the fact, for example, that the individual is complaining of certain discomforts.

Primarily, the information obtained by PC 30 is processed to identify statistically significant deviations from data obtained from a database, which data serves as the norm against which the data derived from the tested individual is compared.

When testing the general state of the individual's health, it is likely that the practitioner will want to measure the voltage spectrum at a given set of sensors placed at specific termination points, and for a specific frequency range. Those voltage patterns are compared to a data set that indicates whether the tested individual is probably in good health, or perhaps not. One data set that is contemplated herein is a data set of past measurements of the individual when that individual was considered healthy. Another data set that is contemplated herein is derived from a collection of measurements of a large group of individuals that are considered healthy. This large group may be undifferentiated, or may be chosen to be most like the tested individual in terms of ethnic background, sex, age, etc. This data can be kept locally (within PC 30) or it can be accessed by PC 30 from a remote location; for example, via the Internet. Of course, if PC 30 communicates the information to a remote computer, it would be advantageous for the data to be co-located with, or easily accessible by, that remote computer.

Illustratively PC 30 receives samples from the set of sensors and analyses the samples to identify the voltage spectra contained in the applied signals. Those voltages, {Vᵢₙₚᵤₜ(f,m)}, where f is the frequency and m identifies the sensor, are compared to corresponding voltages {V_{db}(f,m)} obtained from a database to determine whether voltages {Vᵢₙₚᵤₜ(f,m)} differ to a statistically significant extent from {V_{db}(f,m)}. Methods for determining whether a statistically significant deviation exists between two sets of data are well known in the art. It may be mentioned here that, as disclosed below, the comparing may be to a database signal that is representative of the set {V_{db}(f,m)}.

By comparing the measurements of an individual that is being tested to like measurements of a group of healthy individuals from a class to which the tested individual belongs, a determination is made that the individual is likely to be healthy, or that the measurements of the tested individual deviate to a statistically significant measure from the measurements of healthy individuals. In the latter case the conclusion may be reached that the tested individual may be not healthy.

Additional database data sets are constituted of individuals with specific known maladies. By comparing the measurements of the tested individual against measurements of people who suffer from a particular malady (e.g., people suffering from Crohn's disease) from which the tested individual is suspected to be suffering, a conclusion may be reached as to whether the tested individual is likely suffering from that malady, or not.

Since according to Jing Luo principles each of the organs has numerous appearances in a health-indicating surface (e.g., a person's skin), when it is desired to assess a particular organ it is often advantageous to make the assessment based on the specific set of termination points that provide information about the organ of interest. Indeed, a general evaluation of an individual may simply comprise connecting the individual to a complete set of sensors followed by a series of separate evaluations, each assessing a particular organ by considering a particular subset of the sensors.

In accord with one embodiment, when assessing a particular organ by considering a particular subset of sensors, the measurements obtained from the sensors are combined according to a preselected function, to develop a single combined signal from the sensors' subset. That is, given a set of measurements, vᵢₙₚᵤₜ(f, mₖ) k=1, 2, . . . , K where mₖ identifies the k^{th} sensor and K is the number of employed sensors, the combined signal that represents organ i is

Vₛ₍ᵢ₎(f)=Hᵢ(vᵢₙₚᵤₜ(f,mₖ) k=1, 2, . . . K (1)

A specific example for equation (1) is

$${V}_{s ( i )} ( f ) = {\sum }_{k = 1}^{K} a ( i , {m}_{k} ) {v}_{input} ( f , {m}_{k} ) \qquad ( 2 )$$

where a(i,mₖ) is the sensor selection matrix for organ i composed of a set of multiplicative weights that reflect the strength of the association between the organ i and sensor mₖ.

FIG. 3 presents a flowchart of one method in accord with the principles disclosed herein where weighted sets of the voltage measurements are assessed. The FIG. 3 method assesses a plurality of L organs and, accordingly, at step 42 a set i (which is a row in the aforementioned organ selection matrix) is chosen and control passes to step 43 where Vₛ₍ᵢ₎(f) is evaluated to determine whether it differs to a statistically significant extent from

$${V}_{primary} ( f ) = {\sum }_{k = 1}^{K} a ( i , {m}_{k} ) {v}_{primary} ( f , {m}_{k} ) .$$

In connection with the notion that set i provides weights that are other than zero to the voltages of specific sensors it is noted that one can actually think of the weights themselves as specifying the sets, and having the sum in equation (2) include of all of the sensors, albeit the weight attached to some of the sensors is zero. For example, the set of weights {0, 0.3, 0, 0, 0, 1.25, 0.75, 0} specifies a set that consists of the 2^{nd}, 6^{th} and 7^{th} sensors, with respective weights 0.3, 1.25, and 0.75. Of course, an embodiment where the weights are simply either 1 or 0 is also within the scope of this disclosure, and PC 30 has a conventional user interface that permits a practitioner to set the weights for the different sensors, manually or programmatically, or may simply select an organ that is to be assessed, and the software within PC 30 identifies the appropriate weights for the different sensors.

The results of the step 43 evaluations are stored in step 44 and control passes to step 45 which determines whether all sets have been considered (i=L). If not, the index i is incremented in step 46 and control returns to step 43. When all sets have been considered, control passes to step 47.

The assessments mentioned above may be performed at one particular frequency, at a set of frequencies, or at an entire range of frequencies, either seriatim or concurrently as disclosed above.

Step 47 reviews the results and determines whether, on balance, the state of the different organs as represented by the input data represents a state of good health, or not. In appropriate circumstances, the results of step 47 point to a particular organ that may have a problem, but generally it does not provide a good indication of the specific malady that may exist. That is because the comparison that is made in the above steps is to database data sets that pertain to healthy individuals.

Knowing that something is not quite right with a particular organ (general diagnosis) is of tremendous benefit, but obtaining a specific diagnosis of a malady is better. To that end, the flowchart of FIG. 3 continues to the segment that includes steps 52-57 where better analysis is obtained by correlating the available data with corresponding data of people with a similar spectral presentation of the Jing Luo network and who happen to have a specific problem. Accordingly, in step 52 one of a set of specific termination points (sensors) is chosen—for example, by setting appropriate weight factors or choosing the appropriate sensor selection matrix for that particular malady—and control passes to step 53. At step 53 a database of measurements information is retrieved by PC 30 (either from its own storage or, more likely, from a national database that is remote and accessible to PC 30) and the measurements obtained from the tested individual are correlated to the data in the retrieved database information pertaining to one malady; i.e., a determination is made whether the individual's data deviates from (or is consistent with) the database data of that one malady to a statistically significant extent. Control then passes to step 54 which stores the correlation results and, thence, to step 55, which determines whether all of the M maladies contained in the selected set have been considered. If not, control passes to step 56 where the index j is incremented and control is then returned to step 53 to repeat the correlation relative to the information in a database associated with a different specific malady.

When all of the specific maladies in the set have been considered, control passes from step 55 to step 57, where the results of the correlations may be further assessed. Lastly, control passes to step 60 where the results are reported to the individual and, optionally, the set of measurements that was obtained from the tested individual is forwarded to the database for incorporation and consequent improvement of the database.

Privacy laws may dictate that the newly acquired patient information, and certainly the collection of previously acquired patient information, may not be permanently stored in PC 30. This is not a significant issue, however, because the patient information can be stored in a simple USB drive or a smart card that the patient maintains and provides to the tester as necessary (for example, to measure the individual against the individual's Jing Luo network of a previous time).

It is noted that the measurements spectra as they appear at different termination points (relative to the common “ground” potential) represent different views of the same Jing Luo network. Therefore, in accord with an additional aspect of this disclosure, following a first set of measurements and assessments are made with a particular Jing Luo Applicator/Detector device, another set of measurements is taken using a different Jing Luo Applicator/Detector device, a different “common” point, or both, to thereby get a number of different spectra sets that, in one sense or another, all focus on the particular organ.

The JLAD of FIG. 1 is merely illustrative; not only in the sense that it is a glove rather than a sock or some other device that can be attached to a body part (e.g., chest), but also in that the measuring points need not necessarily be physical electrical contacts (as already disclosed). For example, one JLAD that was used for detecting electromagnetic radiation from a portion of the body is an off-axis parabolic collector, similar to that of a parabolic dish antenna receiving electromagnetic signal from a remote satellite. An advantage of electromagnetic coupling to measuring points is that the measuring points may be other than termination points on a person's skin (for example, the pupil of an eye). As another example of JLAD, one or more acupuncture needles may be used. Also, a particular JLAD may have a mix of different means for coupling to termination points. The circuit design of element 2 would be different, of course, for capacitive, inductive, or electromagnetic sensors, and for minimally invasive sensors such as an acupuncture needle, but such variations in embodiments may be completely conventional in their designs, and are left to be chosen by the person who practices the principles disclosed herein.

The Vᵢₙₚᵤₜ(f,mₖ) information that is developed above is the combined spectrum information, spanning the entire frequency band. However, just as in the field of genetics where it is possible to focus attention on specific chromosome sequences rather than looking at the entire genome, it is possible in the Jing Luo network to focus on a certain group of frequencies or frequency bands. It ought to be noted, also, that while the data discussed above are in the time and the frequency domains, it is possible to employ other domains.

To summarize, the above-disclosed principles that are illustratively embodied in an arrangement where currents pass through an individual by means of K sensors of a glove that is worn by the individual and a return path that is connected, for example, to the individual's ankle. The voltages are digitized by means of A/D converters, and analyzed.

The embodiment disclosed above, particularly relative to FIG. 3, deals with the spectrum of a representative variable as expressed in equation (2), but that is not a requirement. One can deal with sets of spectra, which would require that the database should contain spectra of individual termination points rather that a single combined spectrum, such as the V_{db}(f) described by equation (3) and, of course, additional processing; but such a larger database also offers greater flexibility in the analysis that is performed.

As for the specifics of the database, it includes a plurality of entries, and each entry pertains to a group of one or more individuals that share a particular profile. Illustratively, each entry pertains to the average spectrum of the group. A group may consist solely of males, people under 30, Caucasians, people of Nordic descent, healthy people, people in the lowest 10 percentile in height, people with liver disease, etc. The group can also consist of only the individual under test at some previous time; and, of course, one can have groups with a profile that combines a number of characteristics, such as males under 30 who have diabetes and who live in the US. In other words, the sets that are available in the database enable one to ascertain whether the tested individual is likely in a healthy state of being, and also to pinpoint the likely existence of a particular malady.

The sets that are obtained from the database for the purpose of measurements are based on the characteristic or characteristics that one wishes to assess. A collection chosen when it is desired to ascertain whether the tested individual is healthy is likely to be different from the collection chosen when it desired to ascertain whether the tested individual has arthritis (in contrast to, perhaps, a sprain). In short, the database entry that is selected is based on the profile against which it is desired to test the individual.

The process of creating the database or databases is not a part of this invention but it is expected that measurements that are made as disclosed herein are collected from many practitioners, and are appropriately combined into a database or databases. The more measurements are collected the more confidant will practitioners be in using the database information as a benchmark.

The voltage spectra from a cluster of termination points in close proximity to each other are closely related to each other; which makes sense because the impedances between sensors that are in close proximity to each other tend to be lower than impedances between sensors that are far apart from each other. However, physical distance is not the sole determinative factor, because the Jing Luo network is such that a termination point of a particular organ can appear some distance away. The important point to note is that a sensor may be highly correlated with one or more other sensors relative to a particular organ, and very poorly correlated with the remaining sensors. From an electrical circuit point of view, one can surmise that signals of two Jing Luo termination points between which there is low impedance will be highly correlated (in the sense that voltages at those termination points will be highly correlated) and two termination points between which there is a high impedance generally will be poorly correlated. It may be realized that there can be exceptions, such as when two Jing Luo terminations points emanating from a particular organ may each be at a long distance away from the organ, hence, have a high inter-termination points impedance, but the signals at the two termination points may still have higher correlations with each other than with any other sensors. What that means is that when an organ (e.g., knee) generates an signal that is enhanced in some sense relative to a norm, it is likely that a termination point in proximity of that organ (first termination point) will exhibit this enhanced condition, and so will all of the other termination points that are correlated with the first termination point (relative to the particular organ); albeit to different degrees. It should be kept in mind that the level of correlation between two Jing Luo termination points may be frequency dependent.

A determination as to whether a particular organ is anomalous and a possible determination as to the precise nature of the malady, as disclosed above, are based on analyzing the signals that are generated from an individual's body and considerations of deviations of voltages from the norm to a significant degree. That is, it is possible to confirm that a malady exists in an organ of an individual by identifying the presence of particular signal conditions as they appear at different Jing Luo termination points of the body. Moreover, in accord with the principles of this disclosure, the presentation of the malady in the individual (e.g. in the sense of the individual suffering from the malady) can be ameliorated by applying energy at appropriate termination points, as disclosed below. In the description below, the term “termination point” applies to any area of an individual's body where the state of health of a particular organ is presented and/or where the state of health of a particular organ can be, or appears to be, affected.

To associate particular signal conditions with the presence of a malady in a particular organ of an individual, it is beneficial to model the body of the individual based on observable signals, which in the case of the FIGS. 1 and 2 equipment is voltages, or signals related to the voltages, that are measured by the sensors. In accord with the principles disclosed herein, the modeling is of the spectra of the voltages, and the model that is illustratively employed is Prony's model, which models the Jing Luo network as a sum of damped sinusoids. For a set of N equally spaced time domain data points, Prony's algorithm seeks to find the M most significant damped sinusoids

$${\sum }_{i = 1}^{M} exp ( {α}_{i} + {β}_{i} k Δ t ) cos ( 2 π {f}_{i} k Δ t + {θ}_{i} ) for k = 0 , 1 , … , N - 1 \qquad ( 3 )$$

that best matches the set of data points. The i^{th} damped sinusoid in the above equation is represented by its amplitude a, damping coefficient βᵢ, phase angle θᵢ and frequency fᵢ, Prony's model is believed reasonable for the application disclosed herein at least partly because the Fourier Transform of a decaying sinusoid in the time domain is a mathematical pole in the frequency domain. The Prony model and algorithm are well known in the art; see, for example, Chapter 11, titled “Prony's Method in Digital Spectral Analysis with Applications,” S. Lawrence Marple, Jr., Prentice Hall (1987), ISBN 0-13-214149-3. Other models might also be reasonable to use.

As indicated above, a malady presents itself in the Jing Luo network through the voltages at specific termination points, where the maladies are characterized by their unique signatures. The parameters that are obtained when modeling an individual with the malady, for example, the damping coefficient-frequency-phase angle sets, differ from these of a healthy individual.

The presence of the malady in a patient is confirmed by identifying presence of the malady's signature in the patient, and in accord with the principles disclosed herein amelioration of the malady is affected by nulling out that signature in the patient's Jing Luo network. The rationale is that the malady's condition is improved, at least as felt by the individual, when the Jing Luo network is affected in a healing session by the application of energy at the proper termination points so as to suppress the signature of the malady. For example, if a patient complains of pain in the left knee and the signature of such a pain is, for sake of illustration, an enhanced magnitude of the damped sinusoid at 235 Hz, and if energy is applied to the patient so as to suppress the enhanced magnitude of the 235 Hz damped sinusoid, then the patient's pain will be mitigated. For best effect, the energy is applied to one or more of the termination points (via sensors) where the signature appears, employing adaptive feedback, which is a well-known technique.

When a patient with a perceived malady (for example, arthritic pain in the left knee) presents himself for a healing session, the practitioner may know the signature of the malady, or may consult a database for that signature. The signature information informs the practitioner of the particular termination points where that malady presents itself (for example, immediately above the knee, at the left pinky, and at the left side of the neck), and the level of correlation among these particular termination points. The practitioner might assume that the patient will present a particular malady at the very same termination points that the practitioner expects and with the same correlation of termination points, or the practitioner may take the view that other termination points might also be involved, an act accordingly.

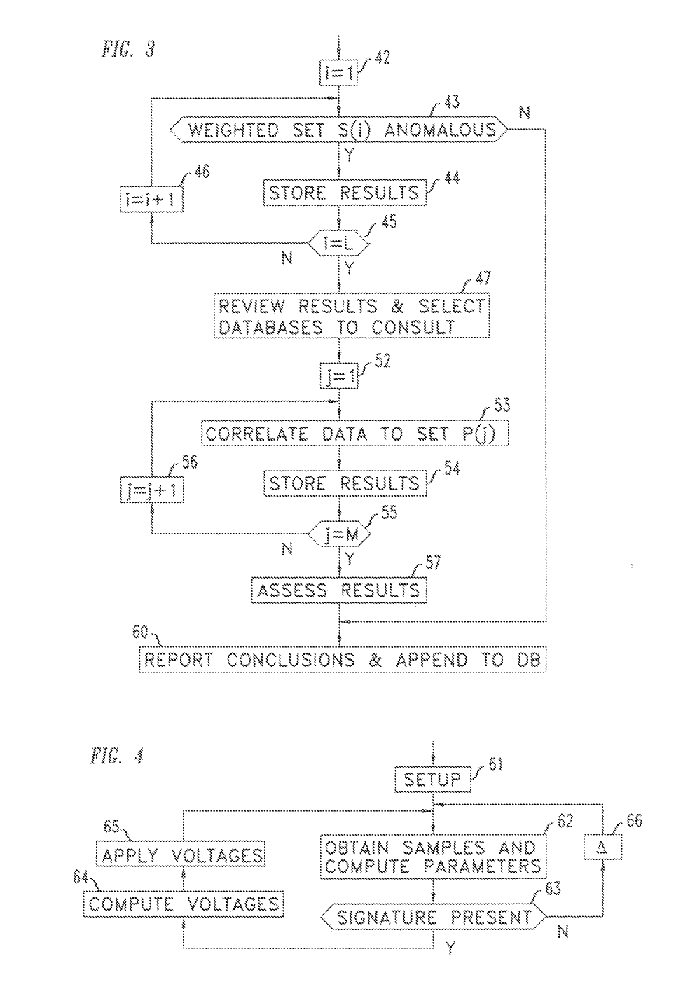

FIG. 4 depicts one illustrative embodiment of a session for ameliorating a malady; for example, an arthritic knee pain. In Step 61 the termination points that are involved with an arthritic knee are identified, and the benchmark set of parameters is retrieved from a database (local or remote). The set includes the termination points where voltages are sensed as well as the termination points where energy is applied. Illustratively, there is a first termination point where voltage is to be applied, and a second termination point where the Jing Luo network is sensed and measured for computing the necessary energy that is to be applied to the first termination point in the adaptive feedback process. It is not necessary to make the set as small as possible because no actual penalty accrues from including termination points that are weakly correlated except for possibly making the modeling process more difficult due to noise contamination in the data, but there is no advantage either in including termination points that are hardly correlated. As practitioners gain experience, it is expected that the number of termination points that they will include in sets will be relatively small.

Control then passes to step 62, where the voltages of the termination points are measured periodically to develop voltage samples. A set of N most recent voltage samples of each termination point are used to deduce the Prony model parameters. This is repeated with each data-taking cycle, except at startup where the processing is delayed until N samples are accumulated.

At step 63, which follows, a determination is made as to whether the parameters computed from the measurements of the second termination points differ to a statistically significant degree from the benchmark parameters. If so, which indicates that the offending enhancement is still present, control passes to step 64. Step 64 computes an energy value, e.g., voltage, that needs to be applied to the first termination point, step 65 applies energy having the computed value, and control returns to step 62 for the next pass through the adaptive feedback loop.

As stated above, step 64 computes the signal (of particular magnitude and frequency spectrum) that specifies the energy that is to be applied to the first termination point based on the voltages at the second termination point. This computation must take into account, however, the fact that whereas the voltage is measured at time t1, the application of energy takes place sometime after the sensing of voltage at the termination points, at time t1+Δ (because of computational delays). What that means, therefore, is that it is advisable to predict the value of at the first termination point at time t1+Δ, v₂^{Δ}(f), from the voltage measurement at time t1, v₂(f). This is accomplished by adding a delay to the phase of the sum of the fitted damped sinusoids in Equation (3), projected to the second termination point. Once v₂^{Δ}(f) is evaluated, if what would be acceptable when the patient is not suffering from the arthritic knee is v̅₂^{Δ}(f), then one needs to apply energy to the first termination point to induce the additive voltage that corresponds to v̅₂^{Δ}(f)−v₂^{Δ}(f) at the second termination point (taking into account the energy that is already present on the patient). Referring to FIG. 2, PC 30 computes the required feedback voltage spectrum to the first termination point. Control passes to step 65 during which PC30 commands the actual generation by the synthesizer 21 through the controller 20, and the feedback energy is applied to the first termination point, and returns control to step 62. It may be noted that the while the time-sampled values of the sensor voltages are obtained by controller 20 at a particular rate, e.g., at a periodicity of T, the processing that takes place in steps 62-65 (Δ) can be longer than Δ. Advantageously, therefore, the sampling rate is limited to no greater than 1/Δ.

It may be noted that there is nothing to prevent the termination points where energy is applied and the termination points where voltage is sensed to be one and the same termination point. While the illustrative example above employs one termination point in an effort to affect another one termination point, a single termination point can be used for both applying energy and measuring voltage, and also multiple termination points can be used to apply energy, with multiple termination points sensed for the effects of the applied energy. It is also not necessary to make the set as small as possible because no actual penalty accrues from including termination points that are weakly correlated except for possibly making the modeling process more difficult due to noise contamination in the data, but there is no advantage either in including termination points that are hardly correlated. As practitioners gain experience, it is expected that the number of termination points that they will include in the set will be relatively small.

The adaptive feedback process of steps 62-65 continues until manually interrupted by the user (for example, after a chosen time duration), or step 63 determines that there are no statistically significant enhancements of the malady's signature and, consequently, passes control to delay step 66 which, following a preselected delay, returns control to step 62. During the loop that includes delay step 66, no changes occur in the applied energies.

In the above discussion, the set of K sensors are treated as a whole for data collection regarding the state of health of the individual as well as recipients of the feedback signals in the malady amelioration session. In fact, the sensors that are used for ameliorating a malady need not necessarily belong to the set of sensors that are used for data collection. For example, the arthritic signature of 253 Hz (in the case of an arthritic left knee) can be masked or eliminated in at least two ways: by applying feedback signals to one or more of the contact sensors, or by applying the feedback signal to the coil placed near the test individual's knee, or third, a combination of the contact sensors and the coil. Due to imperfection of our understanding of the Jing Luo network and of physiology in general, the effects of the two approaches can interfere or correlate with each other. Application of the feedback signal using the coil substantially mitigates the 253 Hz enhancement normally expected at the contact sensors. A simplistic way to look at it is that the coil applies beneficial energy directly to that part of the patient's body that actually generates the 253 Hz enhancement which, in the absence of the beneficial energy would appear at the expected contact sensors. Nulling out the enhancement at the source nulls it out at the correlated termination points.

It may be pointed out that while the above addresses nulling out an enhancement by way of example, it is possible that a deficiency is the cause of a malady, in which case the injected energy will be conditioned to enhance the deficiency. In other words, the notion of “nulling” in the context of this disclosure is the application of energy in an effort to bring a signature toward a desired state.

It is noted that the healing session does not need to be limited in time. Much like a heart pacemaker, one can have a continuous application of energy, as described above. The Jing Luo Applicator/Detector device may be replaced with a Jing Luo collection of devices that an individual may wear for long time durations, or at all times; smart underwear, if you will.

It might be noted also that the methods disclosed above that are executed in the FIG. 2 apparatus are most advantageously controlled by PC 30, under direction of a practitioner and under control of programs that are stored in PC 30. Those programs provide a conventional interface for the practitioner to decide what assessments to conduct, what databases to use in order to obtain the most appropriate benchmarks, what types of sensors are used in the JLAD device, which malady a healing session is dealing with, the duration of a healing session, etc. Other embodiments would be readily apparent to a person who is skilled in the art, without departing from the principles disclosed herein. For example, whatever some or all of the processing disclosed herein may be advantageously offloaded by PC 30 to a remote processor. That processor may, but does not need to be, co-located with the disclosed database.

1. A method comprising the steps of:
developing time-sampled sensor signals from one or more sensors that are respectively coupled to preselected vicinities on a patient's body, each of the sensor signals having a non-sparse frequency spectrum in a particular frequency band;
processing by at least transforming the time-sampled sensor signals to parameters that characterize frequency components contained in respective sensor signals, to form one or more processed signals;
determining whether the processed signals deviate from a database-obtained norm signature to a statistically significant level; and
affecting or informing the patient in response to the determining.

2. The method of claim 1, employed to ameliorate a particular malady of the patient, where said processing performs said transforming, following an initial delay of N sampling intervals, at each sampling interval, by employing a frame, F, of N most recent time-sampled sensor signals, and identifying amplitude, damping coefficient, phase angle, and frequency parameters that best match equation
$$ℱ = {\sum }_{i = 1}^{M} exp ( {α}_{i} + {β}_{i} k Δ t ) cos ( 2 π {f}_{i} k Δ t + {θ}_{i} ) for k = 0 , 1 , … , N - 1$$
where αᵢ, βᵢ, θᵢ, and fᵢ, are the amplitude, damping coefficient, phase angle, and frequency parameters, respectively, and M is a chosen integer.

3. The method of claim 1, employed to ameliorate a particular malady of the patient, further comprising the step of choosing said norm signature from said database to be one that corresponds to a hypothetical patient who is devoid of said malady, or one that corresponds to said patient at an earlier time.

4. The method of claim 3, where
said step of determining also develops energy of particular frequency spectrum, where development of said energy is based on at least one of the processed signals; and
said step of affecting couples said energy to a selected vicinity of the patient's body.

5. The method of claim 3, employed to ameliorate a particular malady of the patient, where
said step of developing time-sampled sensor signals yields a digital voltage representation for each one of said one or more sensors at each interval of a clock;
said step of processing the sensor signals, following a startup interval, takes place at each sampling interval and makes results of said processing available Δ sampling intervals following commencement of said processing;
said step of determining includes computing a measure of energy to be coupled the patient, of particular frequency spectrum, where computation of said energy is based on said one or more of the processed signals;
said step of affecting develops and couples the computed energy to a selected vicinity of the patient's body via a sensor of said sensors; and
said method further comprises a step, following said step of affecting the patient in response to said determining, of repeating turning to said steps of processing, determining and affecting.

6. The method of claim 5, where said step of computing the energy measure predicts said processed signals at said Δ sampling intervals following commencement of said processing.

7. The method of claim 6 where said step of repeating is executed until a preselected condition is met.

8. The method of claim 5 where said step of developing energy takes account of energy present to on said patient at execution time of said step of processing.

9. The method of claim 1, employed to determine state of health of the patient, where said transforming is effected by use of the Fast Fourier Transform algorithm.

10. The method of claim 1, employed to determine state of health of the patient, where the chosen norm signature is related one or more characteristics of the patient.

11. The method of claim 1, employed to determine state of health of a preselected body system of the patient, where the chosen norm signature is related to said preselected body system.

12. The method of claim 1, employed to determine state of health of a preselected body system of the patient, where the acquired time-sampled sensor signals arise from voltages resulting from energy that is applied to at least one of said sensors, said energy having a given magnitudes and a substantially flat non-sparse frequency spectrum in said chosen frequency band of operation.

13. The method of claim 1, employed to assess state of health of the patient's body or a constituent system of the patient's body, further comprising a step of injecting energy to said vicinities, which energy has a given magnitude and a substantially flat non-sparse frequency spectrum in said particular frequency band; where
said sensor signals arise from the injected energy;
said processing transforms said time-sampled sensor signals to frequency domain;
said norm signature is related
to a particular body system that is being assessed, when said particular body system is being assessed, and
to one or more attributes of the patient, taken from a set that includes identity, sex, height, weight, genetic attributes, race attributes, national origin, considered malady that potentially afflicts the patient, and health history of the patient; and
said step of affecting or informing provides a visual presentation reflecting said determining.

14. The method of claim 13 where said processing, in addition to transforming the time-samples sensor signals x(n,mₖ), n=0, 1, . . . (N−1), to the frequency domain and obtaining frequency samples X(fⱼ,mₖ) j=0, 1, . . . , N/2−1, where N is an integer, k=1, 2, . . . , K, combines the frequency samples in accord with a preselected combining function, H, to form a processed signal X_{combined}(f)=H(X(f,m)) of said one or more processed signals.

15. The method of claim 14 where said combining develops said processed signal corresponding to
$${X}_{combined} ( f ) = {\sum }_{k = 1}^{K} a ( {m}_{k} ) X ( f , {m}_{k} ) ,$$
where a(mₖ)>0, k=1, 2, . . . , K are preselected coefficients.

16. The method of claim 14 where said one or more sensors form a set of K sensors, and said combining develops said processed signal in accord with
$${X}_{combined} ( f ) = {\sum }_{k = 1}^{K} a ( {m}_{k} ) X ( f , {m}_{k} ) ,$$
where a(mₖ)=0 for some values of k and otherwise for remaining values of k, where the values of k for which a(mₖ)=0 are dictated by the body system of which the state of health is assessed.

17. Apparatus comprising:
a first module adapted to develop one or more time-sampled signals (signals A) from a set of one or more applied signals;
a second module that is adapted to
process said signals A by at least transforming said signals A to parameters that characterize frequency components contained in said signals A, thereby forming one or more processed signals, where said transforming is adapted to handle said signals A that were developed from said applied signals that each have a non-sparse frequency spectrum in a particular frequency band;
reach a determination regarding extent to which the processed signals deviate to a statistically significant level from a database-obtained norm signature; and
a third module adapted to output a report based on said determination, or output energy with magnitude and frequency spectrum that is computed based on said determination.

18. The apparatus of claim 17 further comprising a module adapted to receive information from a user of said apparatus, which information affects the norm signature that is obtained from the database.

19. The apparatus of claim 17 further comprising
a module for accessing the database in accord with information received from a user of said apparatus or in accord with data that was previously generated in said apparatus.

20. The apparatus of claim 17 where said signature that is obtained corresponds to a hypothetical patient who of particular characteristics, or corresponds to said patient at a specified past time.

21. The apparatus of claim 17 where said third module is adapted to affect said patient by applying amelioration energy to a target vicinity of said patient's body, where the amelioration energy, which said second module is adapted to develop, has a specified frequency spectrum.

22. The apparatus of claim 21 where development of said amelioration energy includes modeling the spectrum of said processed signals.

23. The apparatus of claim 22 where said modeling employs the Prony algorithm.

24. The apparatus of claim 19 where
said second module is adapted to affect said patient by developing a level of energy to be applied to the patient, when the preselected process determines that the patient's signature deviates from said norm signature to a statistically significant level, where said level of energy is based on at least one of the processed signals; and
and said third module is adapted to couple said level of energy to a selected vicinity of the patient's body via a sensor of said sensors.

25. The apparatus of claim 24 where said second module is adapted to cycle through said developing until a preselected condition is met.

26. The apparatus of claim 21 where said second module is adapted to develop, while applying said amelioration energy, a measurement, at a given instant, where said measurement relates to a signal that appears at each of specified one or more of said sensors; and in response to said measurement modifies said amelioration energy in a direction that, at a next instant, causes a change in said measurement, if at all, toward a specified measurement goal.

27. The apparatus of claim 17 where
said first module comprises a plurality of N sensors from which said set of sensors is taken based on said information; and
said signals that are acquired from said set of sensors result from (a) voltages generated in response to actively injected energy into said patient's body, said energy being of a given magnitude and of substantially flat frequency spectrum that spans a preselected bandwidth, or (b) from voltages spontaneously generated by the patient's body.

28. A non-transitory computer readable medium on which is stored a set of machine readable instructions that, when execution of said instructions is requested by a processor, execute the steps of:
accepting an N plurality of signals representative of voltage spectra at N different vicinities on a patient's body;
fetching information from a database that pertains to a specified profile of patients;
forming a determination as to whether said information contained in said signals deviates from said information to a statistically significant level; and
reporting on said determination, or affecting said patient in response to said determination.

29. A non-transitory computer readable medium comprising
a first stored module of machine readable instructions that, when executed by a processing apparatus that is adapted to develop time-sampled sensor signals from signals of a set of one or more sensors, processes the time-sampled sensor signals by at least transforming the time-sampled sensor signals, including when each of the signals of the set of one or more sensors has a non-sparse frequency spectrum in a particular frequency band, to parameters that characterize frequency components contained in the respective sensor signals, to form one or more processed signals,
a second stored module of machine readable instructions that, when executed by said processing apparatus reaches a determination whether the processed signals deviate from a norm signature to a statistically significant level, wherein the norm signature is obtained from a database, and
a second stored module of machine readable instructions that, when executed by said processing apparatus outputs a report based on said determination, or affects a patient in response to said determination when said one or more sensors couple preselected vicinities on a patient's body to said first module.
